(19) United States
(12) Patent Application Publication
(10) Pub. No.: US 2006/0277611 A1
(43) Pub. Date: Dec. 7, 2006

(54) Functional characterization of myo-inositol monophosphatase

(76) Inventors: Wendy Mia Albert Balemans, Kalmthout (DE); Diederik Willem Elisabeth Moechars, Beerse (BE); Thomas Horst Wolfgang Steckler, Mol (BE); Kim Cryns, Beerse (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/557,109

(22) PCT Filed: May 13, 2004
(86) PCT No.: PCT/EP04/05190

§ 371 Date: Nov. 14, 2005

(30) Foreign Application Priority Data
May 16, 2003 (EP) 03/5319

(51) Int. Cl. A01K 67/027; C12Q 1/68; G01N 33/567

(52) U.S. Cl. 800/3; 435/6; 435/7.2; 800/12

(57) ABSTRACT

This invention relates to the functional characterization of myo-inositol monophosphatase 2 (IMPA2), one of the enzymes acting in the phosphatidyl inositol signaling pathway. In particular, the present invention provides evidence that IMPA2 is associated with depression- and anxiety inducing conditions, in particular anxiety and affective disorders. In a first aspect the present invention provides the use of an IMPA2 enzyme in an assay to identify anti-anxiety or an anti-depression compounds. In particular to the use of an isolated polynucleotide encoding said IMPA2 protein, wherein said IMPA2 protein is preferably being selected from polynucleotides encoding the mouse, rat or human IMPA2 enzyme. It is thus an object of the present invention to provide a method for identifying anti-anxiety or anti-depression compounds wherein said compounds are capable of enhancing neuronal plasticity, said method comprising the steps of: a) providing a composition comprising an IMPA2 protein; b) contacting the IMPA2 protein with the test compound; and c) measuring the activity of the IMPA2 protein wherein a decrease in the IMPA2 activity in the presence of the test compound is an indicator of an anti-anxiety or anti-depression compound. In these assays the activity of the IMPA2 protein is assessed by measuring the hydrolysis of myo-inositol 1-phosphate to generate inositol and inorganic phosphate, in particular by measuring the accumulation of either myo-inositol monophosphate product in the form of radiolabeled inositol or inorganic phosphate (Pi) in the form of radiolabeled ³²Pi or in a colorimetric assay. The compositions comprising the IMPA2 protein could either be cellular extracts, whole cells or organisms expressing the IMPA2 proteins according to the invention.

This invention relates to the functional characterization of myo-inositol monophosphatase 2 (IMPA2), one of the enzymes acting in the phosphatidyl inositol signaling pathway. In particular, the present invention provides evidence that IMPA2 is associated with depression- and anxiety inducing conditions.

### BACKGROUND OF THE INVENTION

The myo-inositol monophosphatase (IMPA) enzyme has an important role in the phosphatidylinositol signaling system, catalyzing the dephosphorylation of various myo-inositol monophosphates to free myo-inositol (Berridge and Irvine, 1989). Biochemical studies have shown that lithium exerts an uncompetitive inhibition of the IMPA enzyme, probably by binding to and blocking metal-binding sites in the enzyme. The reduced activity of IMPA may lead to a depletion of intracellular free myo-inositol, which is used in the re-synthesis of the signal precursor inositol phospholipid (Berridge et al., 1989). Lithium has for several decades been used as a mood-stabilizer in the treatment of manic-depressive (bipolar) illness. However, the molecular mechanism of the mood-stabilizing effect has not been established. The inhibition by lithium on IMPA activity and its anti-bipolar effect appear within the same range of concentrations and this biochemical effect remains an intriguing hypothesis for the moods stabilizing action of lithium.

It has been proposed that variations (e.g. loss-of-function or gain-of-function mutations) in the genes encoding myo-inositol monophosphatases could either be implicated in the disturbed neuronal activity of bipolar disorder or explain the observed variations in the therapeutic efficacy of lithium (Steen et al., 1996). So far, two human genes, IMPA1 and IMPA2, have been cloned and predicted to encode IMPA enzymes (McAllister at al. 1992; Sjøholt et al. 2000). Interestingly, the human IMPA2 gene is located on chromosome 18p11.2 a region that in several linkage studies has been indicated as a susceptibility locus for bipolar disorder. Further evidence for a possible association of IMPA2 with bipolar was given in a study of the B lymphoblast cell lines from bipolar I affective disorder (BD-I) patients. It was found that these cells from male BD-I patients have significantly lower IMPA2 mRNA levels and elevated basal intracellular calcium levels compared with healthy male subjects (Yoon et al, 2001).

In a study to explore the possible role of this enzyme as a target for the mood-stabilizing action of lithium in manic-depressive illness, IMPA2 knockout mice were phenotyped using a number of traditional behavioral tests.

The present results indicate that IMPA2 knockout mice are less prone to anxiety and depression-inducing conditions and point to a possible role for IMPA2 in affective disorders, in particular to a role in the impaired neuroplasticity and cellular resilience found in severe mood and anxiety disorders.

The functional characterization by the present invention of IMPA2 as a gene involved in neuronal plasticity provides the means to identify compounds useful in the treatment of patients that have an impaired capability of neuronal cells to make a long term alteration of its circuitery and functionally in response to new inputs (learning), as well as in the treatment of patients that have an impaired capability of the neuronal tissue to recover from injury by reorganizing its function to compensate for partial destruction of tissue or loss of function caused be degenerative disorders.

### SUMMARY OF THE INVENTION

In a first aspect the present invention provides the use of an IMPA2 enzyme in an assay to identify anti-anxiety or anti-depression compounds, wherein said anti-anxiety or anti-depression compounds are capable of enhancing neuronal plasticity. Consequently, in a further aspect the present invention provides the use of an IMPA2 enzyme in an assay to identify compounds capable to enhance the neuronal plasticity in the CNS of a mammal.

It is thus an object of the present invention to provide a method for identifying anti-anxiety or anti-depression compounds wherein said anti-anxiety or anti-depression compounds are capable of enhancing neuronal plasticity, said method comprising the steps of:

a) providing a composition comprising an IMPA2 protein;

b) contacting the IMPA2 protein with the test compound; and

c) measuring the activity of the IMPA2 protein wherein a decrease in the IMPA2 activity in the presence of the test compound is an indicator of an anti-anxiety or anti-depression compound.

In a second aspect, the present invention provides a method for determining whether a compound is a capable of enhancing neuronal plasticity, said method comprising the steps of;

a) providing a composition comprising an IMPA2 protein;

b) contacting the IMPA2 protein with the test compound; and

c) measuring the activity of the IMPA2 protein wherein a decrease in the IMPA2 activity in the presence of the test compound is an indicator of a neuronal plasticity enhancing compound.

In a third aspect, the invention provides the use of a compound identified using an assay according to the invention, in the preparation of a medicament for treating anxiety or in the preparation of a medicament for promoting neuronal plasticity, in particular in the preparation of a medicament to enhance memory or to treat memory dysfunction, as well as to treat neuronal damage of the following kinds: stroke, multi-infarct dementia, head trauma, cerebral ischemia, brain injury, including (without limitation) injury casude by assault, accident, tumour (e.g. a brain tumour or a non-brain tumour that affects the brain, such as bony tumour of the skill that impinges on the brain) or surgery to remove tumours or to treat epilepsy; multiple sclerosis; and neurodegenerative diseases which affect the cortex, such as senile dementia, Alzheimer's disease, Parkinsons's disease, Huntington's chorea, cerebellar-spinal adrenoleucodystrophy, Pick's disease or Wilson's disease.

In a fourth aspect the invention provides a method of treatment of a condition associated with an impaired neuronal adaptive response, such as for example in the treatment of memory dysfunction, as well as to treat neurodegenerative diseases which affect the cortex, such as senile dementia, Alzheimer's disease, Parkinsons's disease, Huntington's chorea, cerebellar-spinal adrenoleucodystrophy, Pick's disease or Wilson's disease, comprising the step of administering an effective amount of an IMPA2 inhibitor to a subject in need of such treatment.

It is also an object of the present invention to provide a method of treating neurological conditions for which neuronal plasticity enhancing treatments are envisaged, such as for example to enhance memory and learning, as well as to treat neuronal damage of the following kinds: stroke, multi-infarct dementia, head trauma, cerebral ischemia, brain injury, including (without limitation) injury casude by assault, accident, tumour (e.g. a brain tumour or a non-brain tumour that affects the brain, such as bony tumour of the skill that impinges on the brain) or surgery to remove tumours or to treat epilepsy; multiple sclerosis; and neurodegenerative diseases which affect the cortex, such as senile dementia, Alzheimer's disease, Parkinsons's disease, Huntington's chorea, cerebellar-spinal adrenoleucodystrophy, Pick's disease or Wilson's disease, comprising the step of administering an effective amount of an IMPA2 inhibitor to a subject in need of such treatment.

In a final aspect, the present invention provides the use of IMPA2 knock out animals as a model to study the effects of enhanced neuronal plasticity. In particular to study the effects of an increased adaptive response to a stressor in an animal model. Such transgenic animals can be commercially marketed to researchers, among other uses.

This and further aspects of the present invention will be discussed in more detail hereinafter.

### BRIEF DESCRIPTION OF THE DRAWINGS

FIG. 1: Schematic representation of the vector VICTR48 used to generate the IMPA2 knockout from OST203987.

FIG. 2: Expression levels of IMPA1 and IMPA2 in different mouse tissue samples. The expression levels in the different mouse tissues are expressed as relative levels after normalization to mouse β-actin.

FIG. 3: Expression levels of IMPA1 and IMPA2 in different mouse tissue samples. The expression levels in the different mouse tissues are expressed as average cycle treshold (CT)-values.

FIG. 4: Results of the different parameters monitored in the Elevated Zero Maze test, i.e. the total distance moved, the relative duration in the open arms and the relative distance in the open arms.

FIG. 5: Results of the different parameters monitored in the first session of the Porsolt forced swim test, i.e. relative immobility duration during the first 180 sec, relative immobility during the last 180 sec and the relative duration of the immobility throughout the test.

FIG. 6: Results of the second session of the Porsolt forced swim test. The same parameters were recorded.

FIG. 7: Results of the different parameters monitored in the Open Field test, i.e. time spent in center, distance travelled in center, total distance travelled, number of moves, duration of moves, number of rearings and duration of rearings

FIG. 8: Expression level of MIP synthase in nonstressed vs. stressed Impa2 KO mice and WT littermates. A significant effect of genotype and of stress on expression levels was found. The expression levels are expressed as relative levels after normalization to mouse β-actin.

FIG. 9: Expression level of BDNF in nonstressed vs. stressed Impa2 KO mice and WT littermates. A significant effect of genotype and of stress on expression levels was found. The expression levels are expressed as relative levels after normalization to mouse β-actin.

This invention will be better understood by reference to the Experimental Details that follow, but those skilled in the art will readily appreciate that these are only illustrative of the invention as described more fully in the claims that follow thereafter. Additionally, throughout this application, various publications are cited. The disclosure of these publications is hereby incorporated by reference into this application to describe more fully the state of the art to which this invention pertains.

### DETAILED DESCRIPTION OF THE INVENTION

In a first aspect the present invention provides the use of an IMPA2 enzyme in an assay to identify anti-anxiety or anti-depression compounds, wherein said anti-anxiety or anti-depression compounds are capable of enhancing neuronal plasticity. Consequently, in a further aspect the present invention provides the use of an IMPA2 enzyme in an assay to identify compounds capable to enhance the neuronal plasticity in the CNS of a mammal. The IMPA2 protein or functional fragment as used herein refers to an isolated protein capable of hydrolysing myo-inositol 1-phosphate to generate inositol and inorganic phosphate. It is preferably selected from the group consisting of;

i. mouse IMPA2 (SEQ ID No:4), rat IMPA2 (SEQ ID No:6), human IMPA2 (SEQ ID No:2) or a functional fragment thereof, or

ii. an amino acid sequence encoding an IMPA2 protein, wherein said amino acid sequence has at least 80% sequence identity, preferably at least 90% sequence identity, more preferably at least 95% or most preferably at least 98% sequence identity with the human IMPA2 protein (SEQ ID No:2) over its entire length.

In particular to the use of an isolated polynucleotide encoding said IMPA2 protein in an assay according to the invention, wherein said IMPA2 protein is preferably being selected from;

i. polynucleotides encoding the mouse (EMBL:BC011093—SEQ ID No:3), rat (EMBL:AY160191—SEQ ID No:5) or human (EMBL:BC011093—SEQ ID No:1) IMPA2 enzyme; or

ii. a polynucleotide sequence encodig an IMPA2 protein, wherein said amino acid sequence has at least 80% sequence identity, preferably at least 90% sequence identity, more preferably at least 95% or most preferably at least 98% sequence identity with the polynucleotide encoding for the human IMPA2 protein (SEQ ID No:1) over its entire length.

“Neuronal plasticity”, “plasticity”, “neuroplasticity” and the like, as used herein refers to the ability of the nervous system to change and/or to develop connections between neurons so as to alter the function of the brain or spinal cord, often in response to sensory or behavioural stimuli or damage. The term encompasses neurogenesis, the activation of synapses that were structurally present but inactive, the strengthening and weakening of synapses, and the making and breaking of synapses.

There are many neurological conditions for which neuronal plasticity enhancing treatments are under investigation. For example, to enhance memory or to treat memory dysfunction, as well as to treat neuronal damage of the following kinds: stroke, multi-infarct dementia, head trauma, cerebral ischemia, brain injury, including (without limitation) injury casude by assault, accident, tumour (e.g. a brain tumour or a non-brain tumour that affects the brain, such as bony tumour of the skill that impinges on the brain) or surgery to remove tumours or to treat epilepsy; multiple sclerosis; and neurodegenerative diseases which affect the cortex, such as senile dementia, Alzheimer's disease, Parkinsons's disease, Huntington's chorea, cerebellar-spinal adrenoleucodystrophy, Pick's disease or Wilson's disease.

It is thus an object of the present invention to provide a method for identifying anti-anxiety or anti-depression compounds wherein said anti-anxiety or anti-depression compounds are capable of enhancing neuronal plasticity, said method comprising the steps of:

a) providing a composition comprising an IMPA2 protein;

b) contacting the IMPA2 protein with the test compound; and

c) measuring the activity of the IMPA2 protein wherein a decrease in the IMPA2 activity in the presence of the test compound is an indicator of an anti-anxiety or anti-depression compound.

In a further aspect, the present invention provides a method for determining whether a compound is a capable of enhancing neuronal plasticity, said method comprising the steps of;

a) providing a composition comprising an IMPA2 protein;

b) contacting the IMPA2 protein with the test compound; and

c) measuring the activity of the IMPA2 protein wherein a decrease in the IMPA2 activity in the presence of the test compound is an indicator of a neuronal plasticity enhancing compound.

The compositions comprising the IMPA2 protein could either be cellular extracts, whole cells or organisms expressing the IMPA2 proteins according to the invention. In a particular embodiment the composition comprising an IMPA2 protein consists of whole cells expressing IMPA2, more particular of CHO cells expressing IMPA2.

Typically the contacting is effected from about 1 minute to about 24 hours, preferably from about 2 minutes to about 1 hour, more preferably the contacting is effected for 1 hour.

In these assays the activity of the IMPA2 protein is assessed by measuring the hydrolysis of myo-inositol 1-phosphate to generate inositol and inorganic phosphate, in particular by measuring the accumulation of either myo-inositol monophosphate product in the form of radiolabeled inositol or inorganic phosphate (Pi) in the form of radiolabeled ³²Pi or in a colorimetric assay. For example, a Pi-release assay based on colorimetric means to measure changes in Pi concentration over time can be carried out as described by Ragan (1988) Biochem. J. 249:143-148, or, by Vadnal (1995) Neuropsychopharmacol. 12:277-285.

As in Vadnal (1995) supra, the reaction mixture can consist of 0.05 ml of 120 mM Tris-HCI, pH 7.8; 0.05 ml of 18 mM or 3 mM magnesium chloride; 0.05 ml of 4.2 mM D-myo-inositol 1-phosphate, 0.125 ml water alone or with positive controls or putative modulator test compounds or compositions. Known myo-inositol monophosphatase inhibitors (antagonists), such as lithium, carbamazepine and/or valproic acid, in varying amounts can be used as controls. A 0.025 ml solution of myo-inositol monophosphatase (e.g., human IMPA2) is added and the reaction mixture is incubated at 37° C. for about 15 minutes to an hour. The reaction is stopped by the addition of 0.05 ml of 20% trichloroacetic acid TCA). The suspension is centrifuged and 0.10 ml of supernatant is used to estimate the liberated Pi using the malachite green reagent method, as, for example, described by Eisenberg (1987) Methods Enzymol. 141:127-143. Protein is assayed using the method of Lowry (1951) J. Biol Chem. 193:265-275.

Assays are usually run in triplicate. Alternatively, as in Ragan (1988) supra, the reaction mixture can be in a final volume of 0.300 ml containing 0.1 mM substrate, 250 mM potassium chloride, 50 mM Tris HCl, pH 8.0, and 3 mM magnesium chloride for period of time from 15 minutes to one hour. Released Pi can be measured colorimetrically using the method of Itaya (1966) Olin. Chem. Acta 14:361-366 (see also Kodama (1986) “The initial phosphate burst in ATP hydrolysis by myosin and subfragment-1 as studied by a modified malachite green method for determination of inorganic phosphate,” J Biochem. (Tokyo) 99:1465-1472). The specific activity of myo-inositol monophosphatase is expressed as nanomoles of phosphate liberated per minute (mU) per milligram protein.

Kinetic activity and assessment of potential modulators of the IMPA2 protein of the invention can also be accomplished in vitro and in vivo by measuring accumulation of the substrate myo-inositol monophosphate (myo-inositol I-phosphate) using, for example, assays described by Atack (1993) J. Neurochem. 60:652-658; or, Ragan (1988) supra. Radiolabeled inositol monophosphate accumulation can be measured in tissue culture cells expressing IMPA2 protein in the presence of putative myo-inositol monophosphatase antagonists, for example, as described by Atack (1993) supra. The tissue culture cells can be genetically manipulated, as described hereinafter, to express the IMPA2 protein of the invention, or fragments or variations thereof.

For example, as described above, CHO cells can be manipulated to express very large amounts of exogenous protein. Specifically, to assess the effect of a putative antagonist or agonist on myo-inositol monophosphatase in vivo, CHO cells are first pre-labeled with ³ H-inositol. Prelabeling involves growing cells to confluence for two days in medium containing radiolabeled inositol (e.g., ¹⁴ C-inositol or ³ H-inositol). If using 3H-inositol, 0.5 uCi/mI 80 Ci/mmol (Amersham International) is used. On the day of the experiment, cells are harvested in Krebs-Henselcit buffer at 2×10⁶ cells/ml containing 0.5 UCi/mI ³ H-inositol.

Aliquots of the harvested cells are incubated for one hour at 37° C. in a shaking water bath in the presence of 10 ul of various concentrations of known enzyme inhibitors and test compounds—putative enzyme modulators. Assays are terminated by addition of 300 ul of 1.0 M TCA and centrifuged. 500 ul of supernatant is washed with water-saturated diethyl ether. The pH is adjusted to about 7.0 using 1 M Tris. The supernatants are then applied to Dowex columns. Columns are washed four times with 5 ml of water to elute free ³ H-inositol; then washed three times with 5 ml of 25 mM ammonium formate to elute beta-glycerophosphates. ³ H-inositol 1-monophosphate is collected by washing the column with 10 ml of 200 mM ammonium phosphate and counted on a scintillation counter.

Alternatively, ¹⁴ C-inositol can be used, as described by Ragan (1988) supra. Inhibition of the myo-inositol monophosphatase will result in increased levels of the substrate myo-inositol monophosphate (myo-inositol 1-phosphate), while activation of the enzyme will result in decreased levels of substrate and increased levels of product (inositol and inorganic phosphate).

Using these assays and variations thereof, the kinetics of the IMPA2 enzyme with and without test modulators (e.g., competitive or non-competitive antagonists) can be analyzed using known methods (e.g., Lineweaver-Burke plots, as used, for example by Lee (1996) Xenobiotica 26: 8′) 1-83) 8); for discussion on enzyme kinetic analysis generally see, for example, Suarez (1997) Proc. Nad. Acad Sci. USA 94:7065-7069; Northrop (1997) Bioorg. Med Chem. 5:641-644); Sterrer (1997) J. Recept. Signal Transduct. Res. 17:511-520).

It is thus an object of the present invention to provide the use of tissue culture cells such as for example CHO or HEK293 cells, genetically manipulated to express IMPA2, in an assay according to the invention. Cells suitable for performing an assay according to the invention are preferably higher eukaryotic cells derived from a multicellular organism and advantageously are mammalian cells. Cells may be transformed by any suitable technique available in the art. A number of techniques, such as calcium phosphate precipitation and electroporation are described in Sambrook et al., (1989) Molecular Biology: A Laboratory Manual, Cold Spring Harbor, which is incorporated herein by reference.

In another aspect, the invention provides the use of a compound identified using an assay according to the invention, in the preparation of a medicament for treating anxiety or in the preparation of a medicament for promoting neuronal plasticity, in particular in the preparation of a medicament to enhance memory or to treat memory dysfunction, as well as to treat neuronal damage of the following kinds: stroke, multi-infarct dementia, head trauma, cerebral ischemia, brain injury, including (without limitation) injury casude by assault, accident, tumour (e.g. a brain tumour or a non-brain tumour that affects the brain, such as bony tumour of the skill that impinges on the brain) or surgery to remove tumours or to treat epilepsy; multiple sclerosis; and neurodegenerative diseases which affect the cortex, such as senile dementia, Alzheimer's disease, Parkinsons's disease, Huntington's chorea, cerebellar-spinal adrenoleucodystrophy, Pick's disease or Wilson's disease.

Methods and pharmaceutical carriers for preparation of pharmaceutical compositions are well known in the art, as set out in textbooks such as Remington's Pharmaceutical Sciences, 20^{th} Edition, Williams & Wilkins, Pensylvania, USA.

The compounds and compositions of the invention may be administered by any suitable route, and the person skilled in the art will readily be able to determine the most suitable route and dose for the condition to be treated. Dosage will be at the discretion of the attended physician or veterinarian, and will be dependent on the state and nature of the condition to be treated, the age and general state of health of the subject to be treated, the route of administration, and any previous treatment which may have been administered. The compound identified as an anti-anxiety compound or a neuroplasticity enhacing compound using an assay according to the invention may optionally be administered in conjunction with one or more other pharmaceutically active agent suitable for the treatment of the condition, i.e. it may be given together, before or after one or more such agents. For example, where the condition involves Alzheimer's disease, the compounds may be used in conjunction with treatment with another agent such as an acetyl-cholinesterase active site inhibitor, for example phenserine, galantamine or tracine.

The carrier or diluent, and other excipients, will depend on the route of administration, and again the person skilled in the art will readily be able to determine the most suitable formulation for each particular case. The compound of the invention may be administered orally, topically, or parenterally in dosage unit formulations containing conventional pharmaceutically acceptable carriers, adjuvants, and vehicles. The term parenteral as used herein includes subcutaneous, intravenous, intramuscular, intrathecal, intracranial, injection or infusion techniques. Of particular interest is administration of the compound to the CNS, through the blood brain barrier. The preferred route of administration will be by direct administration to the CNS, e.g. infusion via canulla or injection. Such administration may be directly into the site of injury, into neighbouring tissues or into the cerebrospinal fluid.

The invention includes various pharmaceutical compositions useful for ameliorating disease. The pharmaceutical compositions according to one embodiment of the invention are prepared by bringing a compound of the invention and optionally one or more other pharmaceutically-active agents or combinations of the compound-of the invention and one or more other pharmaceutically-active agents into a form suitable for administration to a subject, using carriers, excipients and additives or auxiliaries.

Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 20th ed. Williams & Wilkins (2000) and The British National Formulary 43rd ed. (British Medical Association and Royal Pharmaceutical Society of Great Britain, 2002; http://bnf.rhn.net), the contents of which are hereby incorporated by reference. The pH and exact concentration of the various components of the pharmaceutical composition are adjusted according to routine skills in the art. See Goodman and Gilman's The Pharmacological Basis for Therapeutics (7th ed., 1985).

The pharmaceutical compositions are preferably prepared and administered in dosage units. Solid dosage units include tablets, capsules and suppositories. For treatment of a subject, depending on activity of the compound, manner of administration, nature and severity of the disorder, age and body weight of the subject, different daily doses can be used. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

For the purposes of this specification it will be clearly understood that the word “comprising” means “including but not limited to”, and that the word “comprises” has a corresponding meaning.

In a furter aspect the invention provides a method of treatment of a condition associated with an impaired neuronal adaptive response, such as for example in the treatment of memory dysfunction, as well as to treat neurodegenerative diseases which affect the cortex, such as senile dementia, Alzheimer's disease, Parkinsons's disease, Huntington's chorea, cerebellar-spinal adrenoleucodystrophy, Pick's disease or Wilson's disease, comprising the step of administering an effective amount of an IMPA2 inhibitor to a subject in need of such treatment.

It is also an object of the present invention to provide a method of treating neurological conditions for which neuronal plasticity enhancing treatments are envisaged, such as for example to enhance memory and learning, as well as to treat neuronal damage of the following kinds: stroke, multi-infarct dementia, head trauma, cerebral ischemia, brain injury, including (without limitation) injury casude by assault, accident, tumour (e.g. a brain tumour or a non-brain tumour that affects the brain, such as bony tumour of the skill that impinges on the brain) or surgery to remove tumours or to treat epilepsy; multiple sclerosis; and neurodegenerative diseases which affect the cortex, such as senile dementia, Alzheimer's disease, Parkinsons's disease, Huntington's chorea, cerebellar-spinal adrenoleucodystrophy, Pick's disease or Wilson's disease, comprising the step of administering an effective amount of an IMPA2 inhibitor to a subject in need of such treatment.

Generally, the terms “treating”, “treatment” and the like are used herein to mean affecting a subject, tissue or cell to obtain a desired pharmacological and/or physiological effect. The effect may be prophylactic in terms of completey or partially preventing a disease or sign or symptom thereof, and/or may be therapeutic in terms of a partial or complete cure of a disease. “Treating” as used herein covers any treatment of, or prevention of disease in a vertebrate, a mammal, particularly a human, and includes: preventing the disease from occurring in a subject which may be predisposed to the disease, but has not yet been diagnosed as having it, inhibiting the disease, i.e arresting its development; or relieving or ameliorating the effects of the disease, i.e. causing regression of the effects of the disease.

As used herein, the term “effective amount” means an amount of a compound of the present invention effective to yield a desired therapeutic response, for example to prevent or treat a disease which is suspectible to treatment by administration of a pharmaceutical composition comprising a compound of the present invention as active ingredient. The specific “therapeutically effective amount” will be at the discretion of the attendant physician or veterinarian and will of course vary with such factors as the particular condition being treated, the physical condition and clinical history of the subject, the type of animal being treated, the duration of the treatment, the nature of concurrent therapy (if any) and the specific formulations employed.

Also described herein are non-human animals and cells which harbor at least one integrated targeting construct that functionally disrupts an endogenous IMPA2 gene locus in said non-human animal or cell, typically by deleting or mutating a genetic element, e.g. exon sequence, splicing signal, promoter enhancer, that is required for efficient functional expression of the IMPA2 gene product. In this embodiment, a portion of the targeting construct integrates into an essential structural or regulatory element of the endogenous IMPA2 gene locus, thereby functionally disrupting it to generate a null allele. Typically, null alleles are produced by integrating a non-homologous sequence encoding a selectable marker (e.g. a neo gene expression cassette) into an essential structural and/or regulatory sequence of an IMPA2 gene by homologous recombination of the targeting construct homology clamps with endogenous IMPA2 gene sequences, although other strategies may be employed.

Most usually, a targeting construct is transferred by electroporation or microinjection into a totipotent embryonal stem (ES) cell line, such as the murine AB-1 or CCE lines. The targeting construct homologously recombines with endogenous sequences in or flanking an IMPA2 gene locus and functionally disrupts at least one allele of the IMPA2 gene. Typically, homologous recombination of the targeting construct with endogenous IMPA2 locus sequences results in integration of a nonhomologous sequence encoding and expressing a selectable marker, such as neo, usually in the form of a positive selection cassette. The functionally disrupted allele is termed an IMPA2 null allele. ES cells having at least one IMPA2 null allele are selected for by propagating the cells in a medium that permits the preferential propagation of cells expressing the selectable marker. Selected ES cells are examined by PCR analysis and/or Southern blot analysis to verify the presence of a correctly targeted IMPA2 allele. Breeding of nonhuman animals which are heterozygous for a null allele may be performed to produce nonhuman animals homozygous for said null allele, so-called “knockout” animals (Donehower et al. (1992) Nature256: 215; Science256: 1392, incorporated herein by reference). Alternatively, ES cells homozygous for a null allele having an integrated selectable marker can be produced in culture by selection in a medium containing high levels of the selection agent (e.g., G418 or hygromycin). Heterozygosity and/or homozygosity for a correctly targeted null allele can be verified with PCR analysis and/or Southern blot analysis of DNA isolated from an aliquot of a selected ES cell clone and/or from tail biopsies.

Several gene targeting techniques have been described, including but not limited to: co-electroporation, “hit-and-run”, single-crossover integration, and double-crossover recombination (Bradley et al. (1992) Bio/Technology 10: 534). The invention can be practiced using essentially any IMPA2licable homologous gene targeting strategy known in the art. The configuration of a targeting construct depends upon the specific targeting technique chosen. For example, a targeting construct for single-crossover integration or “hit-and-run” targeting need only have a single homology clamp linked to the targeting region, whereas a double-crossover replacement-type targeting construct requires two homology clamps, one flanking each side of the replacement region.

For example and not limitation, a preferred embodiment is a targeting construct comprising, in order: (1) a first homology clamp having a sequence substantially identical to a sequence within about 3 kilobases upstream (i.e., in the direction opposite to the translational reading frame of the exons) of an exon of an endogenous IMPA2 gene, (2) a replacement region comprising a positive selection cassette having a pgkpromoter driving transcription of a neogene, (3) a second homology clamp having a sequence substantially identical to a sequence within about 3 kilobases downstream of said exon of said endogenous IMPA2 gene, and (4) a negative selection cassette, comprising a HSV tkpromoter driving transcription of an HSV tkgene. Such a targeting construct is suitable for double- crossover replacement recombination which deletes a portion of the endogenous IMPA2 locus spanning said exon and replaces it with the replacement region having the positive selection cassette. If the deleted exon is essential for expression of a functional IMPA2 gene product, the resultant exon-depleted allele is functionally disrupted and is termed a null allele.

Targeting constructs of the invention comprise at least one IMPA2 homology clamp linked in polynucleotide linkage (i.e., by phosphodiester bonds) to a targeting region. A homology clamp has a sequence which substantially corresponds to, or is substantially complementary to, an endogenous IMPA2 gene sequence of a nonhuman host animal, and may comprise sequences flanking the IMPA2 gene.

Although no lower or upper size boundaries for recombinogenic homology clamps for gene targeting have been conclusively determined in the art, the best mode for homology clamps is believed to be in the range between about 50 basepairs and several tens of kilobases. Consequently, targeting constructs are generally at least about 50 to 100 nucleotides long, preferably at least about 250 to 500 nucleotides long, more preferably at least about 1000 to 2000 nucleotides long, or longer. Construct homology regions (homology clamps) are generally at least about 50 to 100 bases long, preferably at least about 100 to 500 bases long, and more preferably at least about 750 to 2000 bases long. It is believed that homology regions of about 7 to 8 kilobases in length are preferred, with one preferred embodiment having a first homology region of about 7 kilobases flanking one side of a replacement region and a second homology region of about 1 kilobase flanking the other side of said replacement region. The length of homology (i.e., substantial identity) for a homology region may be selected at the discretion of the practitioner on the basis of the sequence composition and complexity of the endogenous IMPA2 gene target sequence(s) and guidance provided in the art (Hasty et al. (1991) Mol. Cell. Biol. 11: 5586; Shulman et al. (1990) Mol. Cell. Biol. 10: 4466). Targeting constructs have at least one homology region having a sequence that substantially corresponds to, or is substantially complementary to, an endogenous IMPA2 gene sequence (e.g., an exon sequence, an enhancer, a promoter, an intronic sequence, or a flanking sequence within about 3-20 kb of a IMPA2 gene). Such a targeting transgene homology region serves as a template for homologous pairing and recombination with substantially identical endogenous IMPA2 gene sequence(s). In targeting constructs, such homology regions typically flank the replacement region, which is a region of the targeting construct that is to undergo replacement with the targeted endogenous IMPA2 gene sequence (Berinstein et al. (1992) Mol. Cell. Biol. 12: 360). Thus, a segment of the targeting construct flanked by homology regions can replace a segment of an endogenous IMPA2 gene sequence by double-crossover homologous recombination. Homology regions and targeting regions are linked together in conventional linear polynucleotide linkage (5′ to 3′ phosphodiester backbone). Targeting constructs are generally double-stranded DNA molecules, most usually linear.

Without wishing to be bound by any particular theory of homologous recombination or gene conversion, it is believed that in such a double-crossover replacement recombination, a first homologous recombination (e.g., strand exchange, strand pairing, strand scission, strand ligation) between a first targeting construct homology region and a first endogenous IMPA2 gene sequence is accompanied by a second homologous recombination between a second targeting construct homology region and a second endogenous IMPA2 gene sequence, thereby resulting in the portion of the targeting construct that was located between the two homology regions replacing the portion of the endogenous IMPA2 gene that was located between the first and second endogenous IMPA2 gene sequences. For this reason, homology regions are generally used in the same orientation (i.e., the upstream direction is the same for each homology region of a transgene to avoid rearrangements). Double-crossover replacement recombination thus can be used to delete a portion of an endogenous IMPA2 gene and concomitantly transfer a nonhomologous portion (e.g., a neogene expression cassette) into the corresponding chromosomal location. Double-crossover recombination can also be used to add a nonhomologous nortion into an endogenous IMPA2 gene without deleting endogenous chromosomal portions. However, double-crossover recombination can also be employed simply to delete a portion of an endogenous IMPA2 gene sequence without transferring a nonhomologous portion into the endogenous IMPA2 gene (see Jasin et al. (1988) Genes Devel.2:1353). Upstream and/or downstream from the nonhomologous portion may be a gene which provides for identification of whether a double-crossover homologous recombination has occurred; such a gene is typically the HSV tkgene which may be used for negative selection.

Typically, targeting constructs of the invention are used for functionally disrupting endogenous IMPA2 genes and comprise at least two homology regions separated by a nonhomologous sequence which contains an expression cassette encoding a selectable marker, such as neo (Smith and Berg (1984) Cold Spring Harbor Symp. Quant. Biol. 49: 171; Sedivy and Sharp (1989) Proc. Natl. Acad. Sci. (U.S.A.)86: 227; Thomas and Capecchi (1987) op.cit. ). However, some targeting transgenes of the invention may have the homology region(s) flanking only one side of a nonhomologous sequence. Targeting transgenes of the invention may also be of the type referred to in the art as “hit-and-run” or “in-and-out” transgenes (Valancius and Smithies (1991) Mol. Cell. Biol.11: 1402; Donehower et al. (1992) Nature 356: 215; (1991) J. NIH Res. 3: 59; which are incorporated herein by reference).

The positive selection expression cassette encodes a selectable marker which affords a means for selecting cells which have integrated targeting transgene sequences spanning the positive selection expression cassette. The negative selection expression cassette encodes a selectable marker which affords a means for selecting cells which do not have an integrated copy of the negative selection expression cassette. Thus, by a combination positive-negative selection protocol, it is possible to select cells that have undergone homologous replacement recombination and incorporated the portion of the transgene between the homology regions (i.e., the replacement region) into a chromosomal location by selecting for the presence of the positive marker and for the absence of the negative marker.

Preferred expression cassettes for inclusion in the targeting constructs of the invention encode and express a selectable drug resistance marker and/or a HSV thymidine kinase enzyme. Suitable drug resistance genes include, for example: gpt(xanthine-guanine phosphoribosyltransferase), which can be selected for with mycophenolic acid; neo(neomycin phosphotransferase), which can be selected for with G418 or hygromycin; and DFHR (dihydrofolate reductase), which can be selected for with methotrexate (Mulligan and Berg (1981) Proc. Natl. Acad. Sci. (U.S.A.)78: 2072; Southern and Berg (1982) J. Mol. IMPA21. Genet. 1: 327; which are incorporated herein by reference).

Selection for correctly targeted recombinants will generally employ at least positive selection, wherein a nonhomologous expression cassette encodes and expresses a functional protein (e. g., neoor gpt) that confers a selectable phenotype to targeted cells harboring the endogenously integrated expression cassette, so that, by addition of a selection agent (e.g., G418 or mycophenolic acid) such targeted cells have a growth or survival advantage over cells which do not have an integrated expression cassette. It is preferable that selection for correctly targeted homologous recombinants also employ negative selection, so that cells bearing only nonhomologous integration of the transgene are selected against. Typically, such negative selection employs an expression cassette encoding the herpes simplex virus thymidine kinase gene (HSV tk) positioned in the transgene so that it should integrate only by nonhomologous recombination. Such positioning generally is accomplished by linking the HSV tkexpression cassette (or other negative selection cassette) distal to the recombinogenic homology regions so that double-crossover replacement recombination of the homology regions transfers the positive selection expression cassette to a chromosomal location but does not transfer the HSV tkgene (or other negative selection cassette) to a chromosomal location. A nucleoside analog, gancyclovir, which is preferentially toxic to cells expressing HSV tk, can be used as the negative selection agent, as it selects for cells which do not have an integrated HSV tkexpression cassette. FIAU may also be used as a selective agent to select for cells lacking HSV tk.

In order to reduce the background of cells having incorrectly integrated targeting construct sequences, a combination positive-negative selection scheme is typically used (Mansour et al. (1988) op.cit., incorporated herein by reference). Generally, targeting constructs of the invention preferably include: (1) a positive selection expression cassette flanked by two homology regions that are substantially identical to host cell endogenous IMPA2 gene sequences, and (2) a distal negative selection expression cassette. However, targeting constructs which include only a positive selection expression cassette can also be used. Typically, a targeting construct will contain a positive selection expression cassette which includes a neo gene linked downstream (i.e., towards the carboxy-terminus of the encoded polypeptide in translational reading frame orientation) of a promoter such as the HSV tkpromoter or the pgk promoter. More typically, the targeting transgene will also contain a negative selection expression cassette which includes an HSV tkgene linked downstream of a HSV tkpromoter.

It is preferred that targeting constructs of the invention have homology regions that are highly homologous to the predetermined target endogenous DNA sequence(s), preferably isogenic (i.e., identical sequence). Isogenic or nearly isogenic sequences may be obtained by genomic cloning or high-fidelity PCR amplification of genomic DNA from the strain of nonhuman animals which are the source of the ES cells used in the gene targeting procedure.

Vectors containing a targeting construct are typically grown in E. coli and then isolated using standard molecular biology methods, or may be synthesized as oligonucleotides. Direct targeted inactivation which does not require prokaryotic or eukaryotic vectors may also be done. Targeting transgenes can be transferred to host cells by any suitable technique, including microinjection, electroporation, lipofection, biolistics, calcium phosphate precipitation, and viral-based vectors, among others. Other methods used to transform mammalian cells include the use of Polybrene, protoplast fusion, and others (see, generally, Sambrook et al. Molecular Cloning: A Laboratory Manual, 2d ed., 1989, Cold Spring Harbor Laboratory Press, Cold Spring Harbor, N.Y., which is incorporated herein by reference).

It is thus a further object of the present invention to provide the use of the IMPA2 knock out animals as a model for neuroplasticity, in particular to study the effects of enhanced neuronal plasticity. In particular to study the effects of an increased adaptive response to a stressor in an animal model. Such transgenic animals can be commercially marketed to researchers, among other uses. The “knock out animal” or “transgenic animal” as used herein refers to a non-human animal, usually a mammal and in particular a rodent, mice, having a 20- non-endogenous (i.e. heterologous) nucleic acid sequence present as an extrachromosomal element in a portion of its cells or stably integrated into its germ line DNA. This heterologous nucleic acid is introduced into the germ line of said transgenic animal by genetic manipulation of, for example, embryos or embryonic stem cells of the host animal using art known procedures.

This and further aspects of the present invention will be discussed in more detail hereinafter.

Experimental

Material and Methods

IMPA2 Knockout Mice

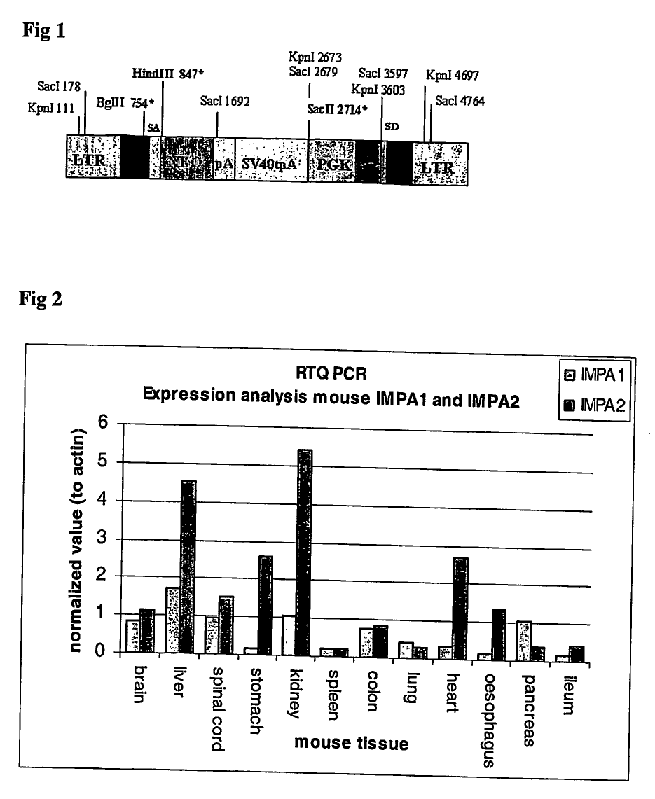

IMPA2 knockout mice were obtained from Lexicon Genetics Inc. and were generated from OST203987. The gene-trap was established with vector VICTR48 and insertion occurred within intron 1 (FIG. 1).

Real-Time Quantitative Reverse Transcription (RTQ) PCR Analysis of IMPA1 and IMPA2.

Total RNA isolated from different tissues dissected from wild type mouse (brain, liver, spinal cord, stomach, kidney, spleen, colon, lung, heart, oesophagus, pancreas, ileum) were analysed using real time quantitative PCR analysis for the tissue distribution of IMPA1 and IMPA2.

First strand cDNA synthesis was performed on 1 μg total RNA using random hexamer primers and Superscript II RT (Invitrogen Life Technologies). Quantitative PCR was performed on an ABIPrism 7000 cycler (Applied Biosystems) using a Taqman PCR kit. Serial dilutions of cDNA were used to generate standard curves of treshold cycles versus the logarithms of concentrations for β-actin. The RTQ specific primer pairs and probes are enlisted herein below.

Mouse IMPA2 Selection of RTQ primers and probe:

| | |
|---|---|
| IMPA2_YW | |
| 5′-GAG GTG GCC GTG CAG TTG-3′ | (SEQ ID No. 7) |
| IMPA2_REV | |
| 5′-AGA CGC GTT TTT CCT CTG TCA-3′ | (SEQ ID No. 8) |
| IMPA2_Probe | |
| 5′-CCT GAT GAT TTG TCC CGC ACG CA-3′ | (SEQ ID No. 9) |
| [5′ FAM] [3′ TAMRA] | |

Mouse IMPA1 Selection of RTQ primers and probe:

| | |
|---|---|
| IMPA1_FW | |
| 5′-AGC TGT TTC AAT TGG CTT CCT T- | (SEQ ID No. 10) |
| 3′ | |
| IMPA1_REV | |
| 5′-GCC GGT GTA CAT CTT ATC TTC CA- | (SEQ ID No. 11) |
| 3′ | |
| IMPA1_Probe | |
| 5′-TGA ATA AAG AGA TGG AGT TTG GAA | (SEQ ID No. 12) |
| TTG TGT ACA GCT-3′ [5′ FAM] | |
| [3′ TAMRA] | |

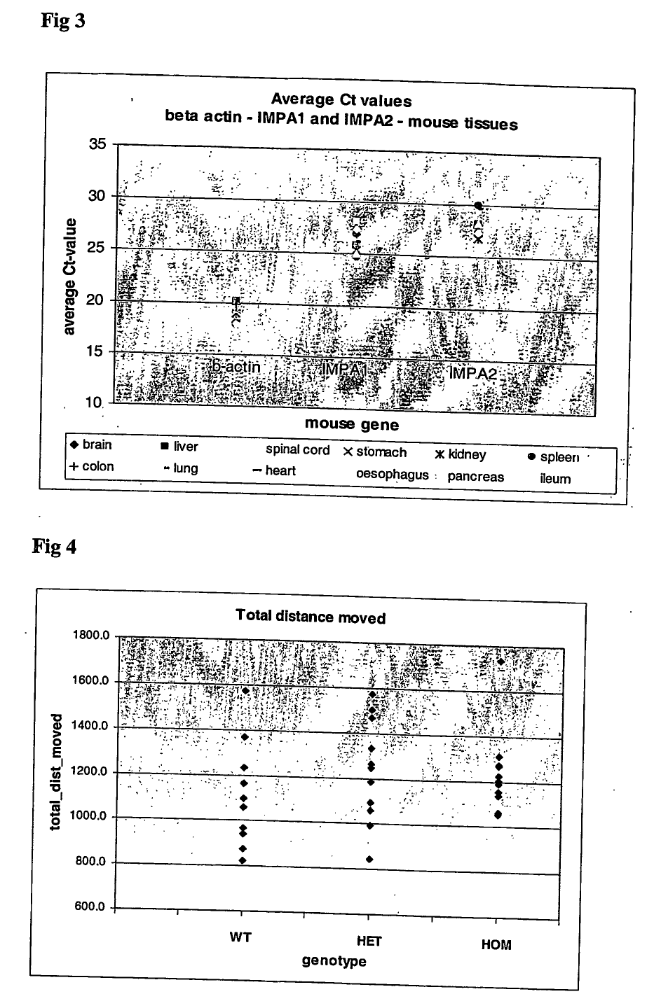

Samples were run in triplicate and results are displayed only when complying with quality standards. Expression levels in the different mouse tissues are expressed as relative levels after normalisation to mouse β-actin (FIG. 2) and as average CT-values (FIG. 3).

Phenotypical Analysis: Mouse Behavioural Tests

A panel of 10 wild type (+/+), 12 heterozygous (+/−) and 11 homozygous (−/−) IMPA2 mice, a mixture of males and females, were subjected to three behavioural tests: Elevated Zero Maze (an art known method to evaluate anxiety-related behaviour), Porsolt Forced Swim test (an art known method to evaluate depression-related behaviour) and Open Field Test (an art known method to evaluate locomotor activity).

Elevated Zero Maze

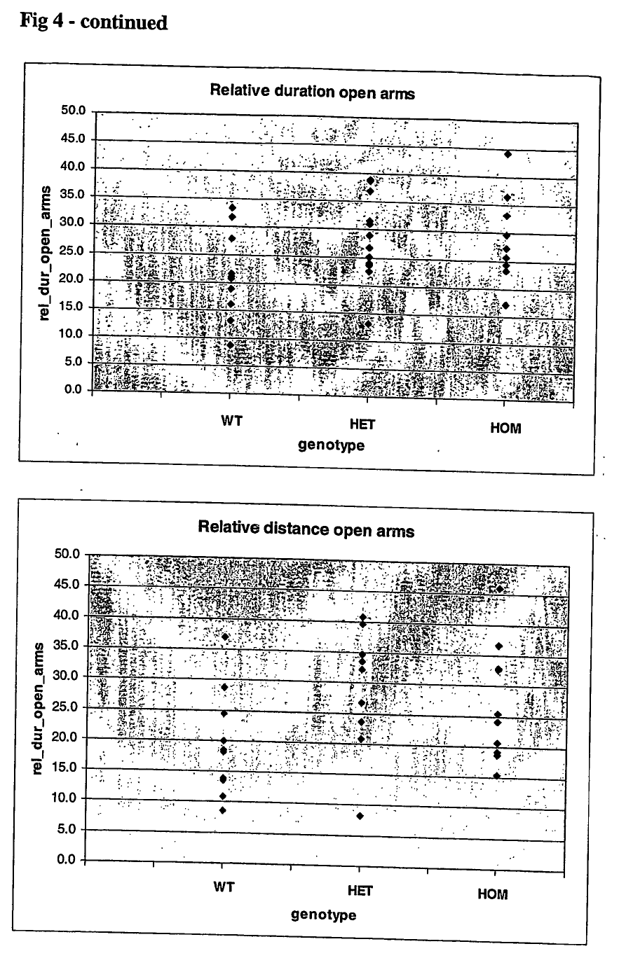

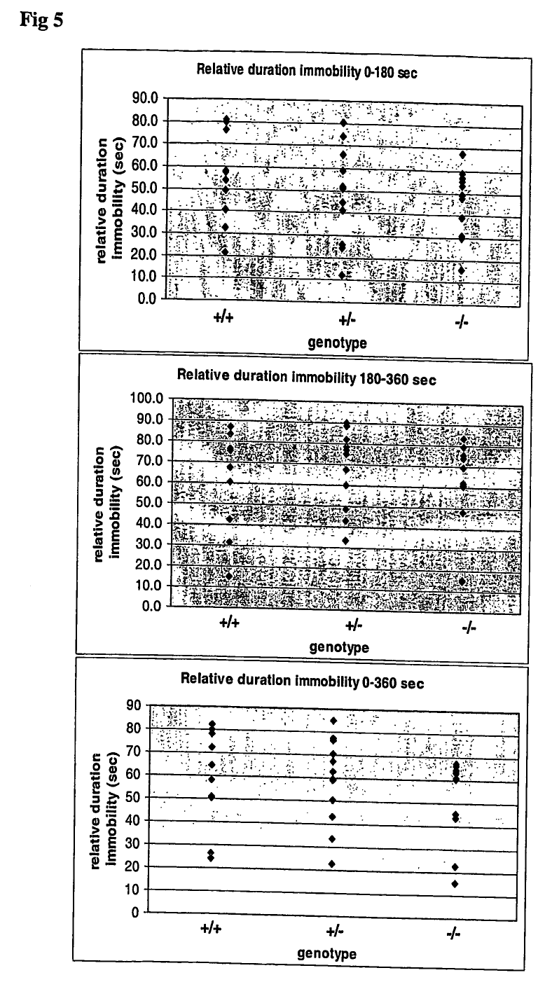

The Elevated Zero Maze was performed during the dark phase of a normal light/dark cyclus. Each mouse was subjected to a 6-min testing session in the elevated zero maze. During this session, the following parameters were recorded: total distance moved, relative distance in the open arms and relative duration in the open arms (FIG. 4). An unadjusted Wilcoxon-Mann-Whitney rank sum test was used as statistical analysis of the data obtained (table 1).

TABLE 1
Descriptive statistics and p-values obtained using an
unadjusted Wilcoxon-Mann-Whitney rank sum test for the
data from the elevated zero maze in the IMPA2 mice

| Parameter | Genotype | N | Mean | St Dev | Median |
|---|---|---|---|---|---|
| Total distance moved | +/+ | 10 | 1111 | 233 | 1078 |
| | +/− | 12 | 1200 | 245 | 1214 |
| | −/− | 11 | 1237 | 185 | 1201 |

| Parameter | Group 1 | Group 2 | P-value |
|---|---|---|---|
| Total distance moved | +/+ | +/− | 0.381 |
| | +/+ | −/− | 0.1517 |

| Parameter | Genotype | N | Mean | St Dev | Median |
|---|---|---|---|---|---|
| Relative distance open arms | +/+ | 10 | 19.35 | 8.64 | 18.28 |
| | +/− | 12 | 28.25 | 9.36 | 29.46 |
| | −/− | 11 | 26.55 | 9.4 | 24.09 |

| Parameter | Group 1 | Group 2 | P-value |
|---|---|---|---|
| Relative distance open arms | +/+ | +/− | 0.0358 |
| | +/+ | −/− | 0.061 |

| Parameter | Genotype | N | Mean | St Dev | Median |
|---|---|---|---|---|---|
| Relative duration open arms | +/+ | 10 | 21.26 | 7.89 | 20.81 |
| | +/− | 12 | 28.41 | 7.58 | 27.85 |
| | −/− | 11 | 29.57 | 7.57 | 27.39 |

| Parameter | Group 1 | Group 2 | P-value |
|---|---|---|---|
| Relative duration open arms | +/+ | +/− | 0.0426 |
| | +/+ | −/− | 0.0357 |

Porsolt Forced Swim Test

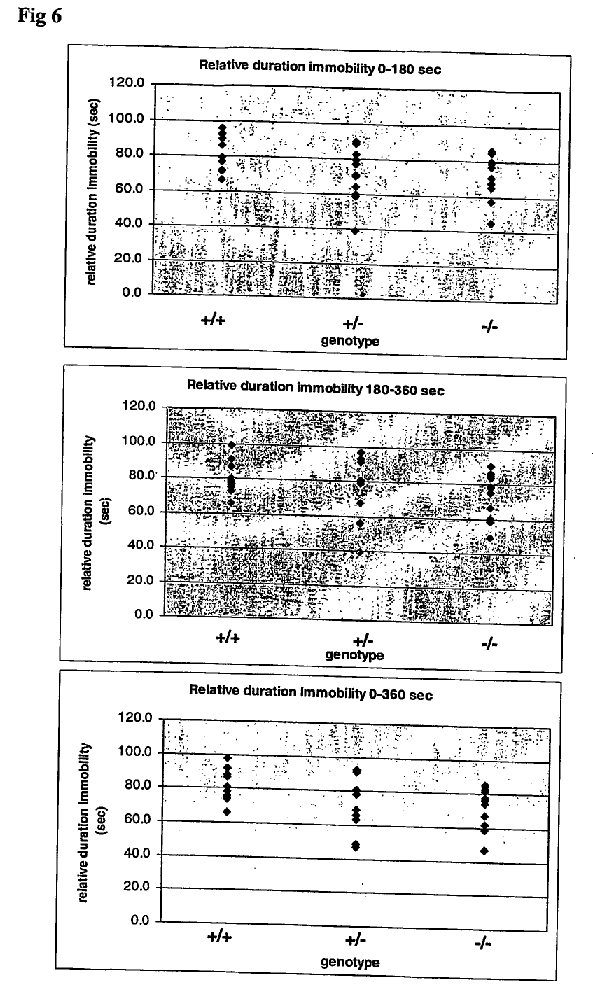

The Porsolt Forced Swim test was carried out over 2 days. On the first day, IMPA2 mice were subjected to a 10-minutes swimming session, of which the first 6 minutes were recorded. 24 hours later, the same mice underwent a second swimming session, this time for 6 minutes, of which 6 minutes were recorded. Each recording period is divided in two separate time intervals: 0→180 sec and 180→360 sec. The Porsolt Forced Swim test was performed during the light phase of a normal light/dark cyclus. During the two sessions, the following parameters were recorded: immobility duration during the first 180 sec and immobility duration during the last 180 sec (FIGS. 5 and 6). An unadjusted Wilcoxon-Mann-Whitney rank sum test was used as statistical analysis of the data obtained (table 2 and 3).

TABLE 2
Descriptive statistics and p-values obtained using an unadjusted
Wilcoxon-Mann-Whitney rank sum test for the data from the
Forced Swim test in the IMPA2 mice on day 1

| Parameter | Genotype | N | Mean | St Dev | Median |
|---|---|---|---|---|---|
| relative immo duration | +/+ | 10 | 55.13 | 20.08 | 55.86 |
| 0-180 sec | +/− | 12 | 48.56 | 20.41 | 51.06 |
| | −/− | 11 | 45.96 | 15.38 | 49.44 |

| Parameter | Group 1 | Group 2 | P-value |
|---|---|---|---|
| relative immo duration | +/+ | +/− | 0.539 |
| 0-180 sec | +/+ | −/− | 0.259 |

| Parameter | Genotype | N | Mean | St Dev | Median |
|---|---|---|---|---|---|
| Relative immo duration | +/+ | 10 | 62.33 | 24.64 | 71.36 |
| 180-360 sec | +/− | 12 | 69.46 | 19.17 | 75.94 |
| | −/− | 11 | 58.57 | 23.69 | 62.17 |

| Parameter | Group 1 | Group 2 | P-value |
|---|---|---|---|
| Relative immo duration | +/+ | +/− | 0.445 |
| 180-360 sec | +/+ | −/− | 0.523 |

| Parameter | Genotype | N | Mean | St Dev | Median |
|---|---|---|---|---|---|
| Relative immo duration | +/+ | 10 | 58.73 | 21.05 | 61.56 |
| 0-360 sec | +/− | 12 | 59.01 | 18.6 | 61.08 |
| | −/− | 11 | 52.27 | 18.23 | 61.08 |

| Parameter | Group 1 | Group 2 | P-value |
|---|---|---|---|
| Relative immo duration | +/+ | +/− | 0.923 |
| 0-360 sec | +/+ | −/− | 0.349 |

TABLE 3
Descriptive statistics and p-values obtained using an unadjusted
Wilcoxon-Mann-Whitney rank sum test for the data from the
Forced Swim test in the IMPA2 mice on day 2

| Parameter | Genotype | N | Mean | St Dev | Median |
|---|---|---|---|---|---|
| relative immo duration | +/+ | 10 | 82.31 | 10.49 | 82.72 |
| 0-180 sec | +/− | 12 | 70.75 | 14.41 | 70.94 |
| | −/− | 11 | 71.28 | 12.33 | 70.67 |

| Parameter | Group 1 | Group 2 | P-value |
|---|---|---|---|
| relative immo duration | +/+ | +/− | 0.0375 |
| 0-180 sec | +/+ | −/− | 0.0513 |

| Parameter | Genotype | N | Mean | St Dev | Median |
|---|---|---|---|---|---|
| Relative immo duration | +/+ | 10 | 80.27 | 9.59 | 77.97 |
| 180-360 sec | +/− | 12 | 73.8 | 17.1 | 79.03 |
| | −/− | 11 | 73.75 | 13.31 | 77.97 |

| Parameter | Group 1 | Group 2 | P-value |
|---|---|---|---|
| Relative immo duration | +/+ | +/− | 0.7223 |
| 180-360 sec | +/+ | −/− | 0.4679 |

| Parameter | Genotype | N | Mean | St Dev | Median |
|---|---|---|---|---|---|
| Relative immo duration | +/+ | 10 | 81.29 | 9.55 | 79.46 |
| 0-360 sec | +/− | 12 | 72.28 | 14.56 | 73.78 |
| | −/− | 11 | 72.52 | 12.36 | 76.14 |

| Parameter | Group 1 | Group 2 | P-value |
|---|---|---|---|
| Relative immo duration | +/+ | +/− | 0.203 |
| 0-360 sec | +/+ | −/− | 0.1734 |

Open Field Test

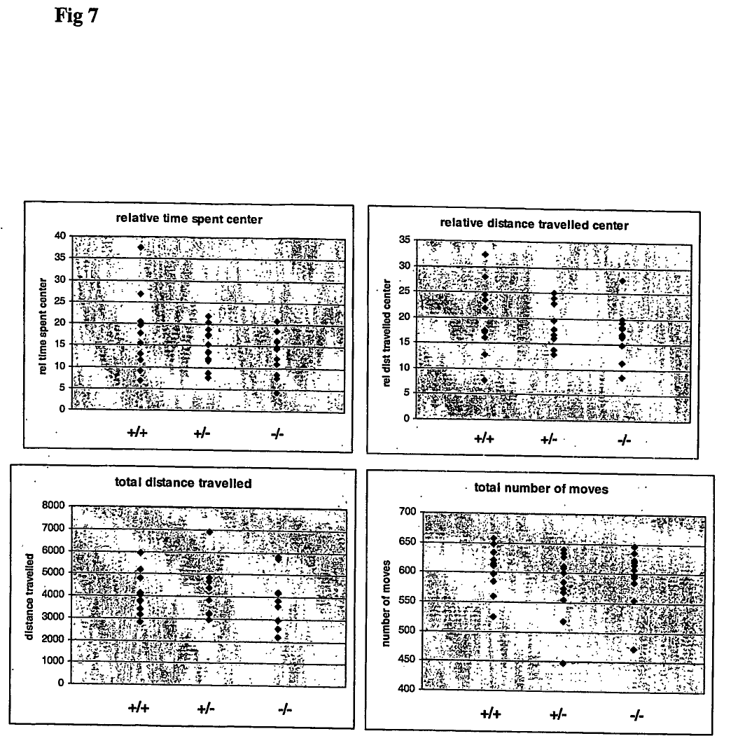

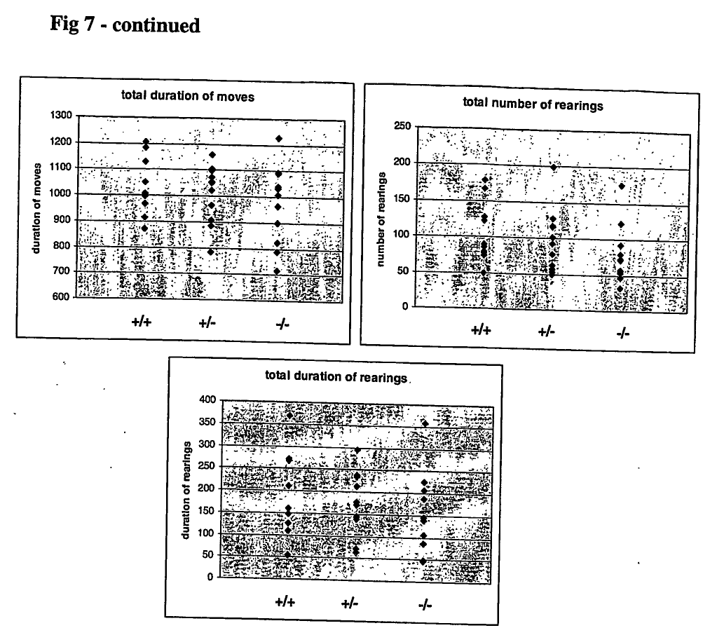

The Open Field Test was performed during the light phase of a normal light/dark cyclus. Each mouse was subjected to a 30-min testing session in an automated open field system. Locomotion in the horizontal and vertical pane was recorded. During this session, the following parameters were recorded: time spent in center, distance travelled in center, total distance travelled, number of moves, duration of moves, number of rearings and duration of rearings (FIG. 7). An unadjusted Wilcoxon-Mann-Whitney rank sum test was used as statistical analysis of the data obtained (table 4).

TABLE 4
Descriptive statistics and p-values obtained using an
unadjusted Wilcoxon-Mann-Whitney rank sum test for the
data from the Open Field test in the IMPA2 mice

| Parameter | Genotype | N | Mean | St Dev | Median |
|---|---|---|---|---|---|
| relative time spent | +/+ | 10 | 17.82 | 9.072 | 16.6 |
| center | +/− | 12 | 14.8 | 4.5 | 14.4 |
| | −/− | 11 | 13.13 | 4.95 | 14.36 |

| Parameter | Group 1 | Group 2 | P-value |
|---|---|---|---|
| relative time spent | +/+ | +/− | 0.5387 |
| center | +/+ | −/− | 0.2816 |

| Parameter | Genotype | N | Mean | St Dev | Median |
|---|---|---|---|---|---|
| relative distance | +/+ | 10 | 20.13 | 7.33 | 19.69 |
| travelled center | +/− | 12 | 18.29 | 3.85 | 16.83 |
| | −/− | 11 | 17.13 | 4.88 | 17.06 |

| Parameter | Group 1 | Group 2 | P-value |
|---|---|---|---|
| relative distance | +/+ | +/− | 0.5387 |
| travelled center | +/+ | −/− | 0.3494 |

| Parameter | Genotype | N | Mean | St Dev | Median |
|---|---|---|---|---|---|
| total distance travelled | +/+ | 10 | 4099 | 960 | 3925 |
| | +/− | 12 | 4207 | 1047 | 4113 |
| | −/− | 11 | 3801 | 1164 | 3820 |

| Parameter | Group 1 | Group 2 | P-value |
|---|---|---|---|
| total distance travelled | +/+ | +/− | 0.7223 |
| | +/+ | −/− | 0.6047 |

| Parameter | Genotype | N | Mean | St Dev | Median |
|---|---|---|---|---|---|
| total number of moves | +/+ | 10 | 605.2 | 40.58 | 613.5 |
| | +/− | 12 | 580.4 | 55.06 | 592 |
| | −/− | 11 | 595.1 | 48.01 | 607 |

| Parameter | Group 1 | Group 2 | P-value |
|---|---|---|---|
| total number of moves | +/+ | +/− | 0.2473 |
| | +/+ | −/− | 0.6415 |

| Parameter | Genotype | N | Mean | St Dev | Median |
|---|---|---|---|---|---|
| total duration of moves | +/+ | 10 | 1034 | 110.5 | 1006 |
| | +/− | 12 | 1013 | 107.6 | 1052 |
| | −/− | 11 | 971 | 152.7 | 1009 |

| Parameter | Group 1 | Group 2 | P-value |
|---|---|---|---|
| total duration of moves | +/+ | +/− | 0.9742 |
| | +/+ | −/− | 0.5116 |

| Parameter | Genotype | N | Mean | St Dev | Median |
|---|---|---|---|---|---|
| total number of rearings | +/+ | 10 | 105.3 | 42.3 | 88 |
| | +/− | 12 | 96.42 | 42.04 | 97 |
| | −/− | 11 | 78.36 | 40.48 | 71 |

| Parameter | Group 1 | Group 2 | P-value |
|---|---|---|---|
| total number of rearings | +/+ | +/− | 0.7105 |
| | +/+ | −/− | 0.0877 |

| Parameter | Genotype | N | Mean | St Dev | Median |
|---|---|---|---|---|---|
| total duration of rearings | +/+ | 10 | 186.6 | 92.89 | 154 |
| | +/− | 12 | 163.7 | 72.86 | 158.5 |
| | −/− | 11 | 150.2 | 91.18 | 139.5 |

| Parameter | Group 1 | Group 2 | P-value |
|---|---|---|---|
| total duration of rearings | +/+ | +/− | 0.7223 |
| | +/+ | −/− | 0.223 |

Real-Time Quantitative Reverse Transcription (RTQ) PCR Analysis of MIP-synthas and BDNF in Non-stressed Versus Mild Stressed Impa2 KO Mice and Wild Type Littermates.

Impa2 KO mice and WT littermates were subjected to restrained stress for 7 consecutive days. On day 1, mice were stressed for 6 hours. On day 2 to 7, mice were stressed for 1 hour daily. On day 8, mice were decapitated, brains were removed and the hippocampus was carefully dissected out. WT and KO mice not subjected to stress were used as controls. Total RNA was isolated from hippocampus. RTQ PCR was performed as described in paragraph ‘Real time quantitative reverse transcription (RTQ) PCR analysis of IMPA1 and IMPA2.’

The RTQ specific primer pairs and probes are enlisted below.

| | |
|---|---|
| MIP synthase_FW | |
| 5′-CTGCGCCTTCCTCAATGG-3′ | (SEQ ID No. 13) |
| MIP synthase REV | |
| 5′-GCTGCGAAGCCAGTTCCA-3′ | (SEQ ID No. 14) |
| MIP synthase_Probe | |
| 5′-TCCCCACAGAACACACTGGTACCCG | (SEQ ID No. 15) |
| [5′]6_FAM [3′]TAMRA | |
| BDNF_FW | |
| 5′-CGGGACGGTCACAGTCCTA-3′ | (SEQ ID No. 16) |
| BDNF_REV | |
| 5′-CACTTGGTCTCGTAGAAATACTGCTT-3′ | (SEQ ID No. 17) |
| BDNF_Probe | |
| 5′-AGAAAGTCCCGGTATCCAAAGGCCAAC | (SEQ ID No. 18) |
| [5′]FAM[3′]TAMRA | |

A Two Way Analysis of Variance was used to analyze the data obtained, followed by Wilcoxon-Mann-Whitney rank sum post hoc analysis. In case data were not normally distributed, data were normalized by log-transformation.

Results

Real-Time Quantitative Reverse Transcription PCR Analysis of IMPA1 and IMPA2.

Real-Time Quantitative Reverse Transcription PCR of IMPA2 and IMPA1 in multiple mouse tissues showed a wide tissue distribution, including brain (FIG. 2). Additionally, expression levels of mouse IMPA1 are generally higher than expression levels of mouse IMPA2 (FIG. 3).

Real-Time Quantitative Reverse Transcription (RTQ) PCR Analysis of MIP-synthas and BDNF in Non-stressed Versus Mild Stressed Impa2 KO Mice and Wild Type Littermates.

It was found that MIP synthase expression levels are higher in IMPA2 KO mice compared to the WT littermates. Further, there is a significant up-regulation of MIP synthase in both the IMPA2 KO mice and the WT littermates in response to stress (FIG. 8).

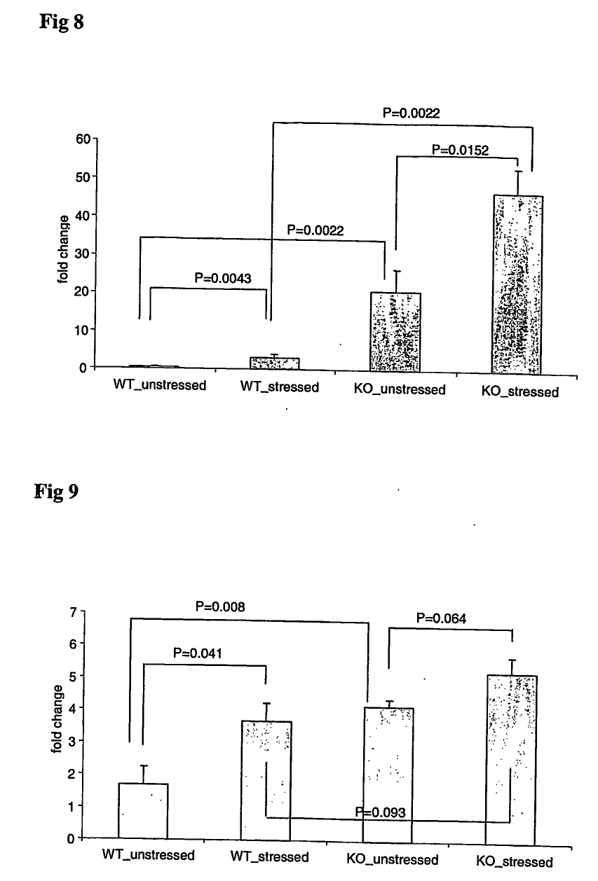

Also for BDNF the genotypic expression levels are higher in IMPA2 KO mice compared to WT littermates and is there a significant up-regulation in stressed versus non-stressed animals (FIG. 9).

Neither for MIP synthase, nor for BDNF, a significant synergy between genotypic expression levels and stress induced expression levels was found.

Phenotypical Analysis: Mouse Behavioural Tests

Elevated Zero Maze

A significant increase in the relative distance traveled in the open arms is observed in the IMPA2 +/− mice compared to their wild type littermates (p=0.0358). We also see a trend in an increased relative distance traveled in the open arms in the IMPA2 −/− mice compared to their WT littermates (p=0.061). Under the anxiety-inducing conditions of the elevated zero-maze, IMPA2 −/− and +/− mice spent a significant longer time in the “anxiogenic” open arms compared to their WT littermates (resp. p=0.0357 and p=0.0426).

Porsolt Forced Swim Test

In the stress-inducing conditions of the forced swim test, no significant differences were observed between IMPA2 −/−, +/− and WT littermates during the first 6 min of a 10-min swim session on the first recording day (table 2). However, this stressful experience on day 1 rendered the IMPA2 −/− and +/− mice with an advantage on the second recording day. I.e. a significant decrease in immobility time was observed in the IMPA2 −/− and +/− mice compared to their WT littermates during the first 3 min of a 6-min swim session (resp. p=0.0513 and p=0.0375; table 3).

Open Field Test

No significant differences were found between IMPA2 −/−, +/− and WT littermates in an open field test for all parameters studied.

Discussion

Myo-inositol monophosphatase 2 (IMPA2) is one of the key enzymes acting in the phosphatidyl inositol signalling pathway. Lithium, the simplest mood-stabilizing drug, inhibits both IMPA1 and IMPA2, key enzymes in the synthesis and recycling of inositol. Additionally, a susceptibility locus for bipolar disorder is mapped on chromosome 18 p, in the region where IMPA2 is located. To further evaluate a potential biological role of IMPA2 in the field of affective spectrum disorders, an IMPA2 knock out was generated (Lexicon Genetics Inc.) and evaluated.

Real-Time quantitative reverse transcription PCR of IMPA2 in multiple mouse tissues showed a wide tissue distribution, including brain. However, expression levels are relatively low compared to IMPA1 levels. Anxiety- and depression-related behaviour of IMPA2-deficient mice were tested in an open field, an elevated zero-maze and in forced swimming. A gene-dosage effect was seen in the zero maze, where IMPA2 −/− mice spent more time on the open areas of the maze than the WT littermates, but did not differ in locomotor activity. No significant differences were observed between IMPA2 −/−, +/− and WT littermates during a first forced swimming session, but a significant decrease in immobility was observed in the IMPA2 −/− and +/− mice compared to the WT littermates during a second session 24 h later. No differences were found between IMPA2 −/−, +/− and WT littermates exploring an open field for 30 min.

In summary, the results presented here indicate that IMPA2 knockout mice show reduced anxiety- and depression-related behaviour, but do not differ from WT littermates in locomotor function and point to a possible role for IMPA2 in affective disorders.

To confirm the initial hypothesis that IMPA2 may have a role in affective disorders, i.e. IMPA2 KO mice suggested a mild antidepressant and anxiolytic phenotype, a further molecular characterization was carried out by studying hippocampal expression changes of MIP synthase and BNDF. MIP synthase is a gene involved in the inositol signaling pathway known to be play a role in manic depression and BDNF is a gene in the neurotrophic signaling pathways, known to be involved in appetitive behaviour and in the development of a depression-like phenotype.

The stress induced up-regulation of MIP synthase and BDNF in IMPA2 KO mice and WT littermates together with the genotypic upregulation in IMPA2 KO, support a role for both the inositol and neurotrophic pathway in the adaptive response to a stressor and suggest that the IMPA2 KO has an improved adaptive potential (non-stressed condition) and response to a stressor.

### References

Berridge, M. J. and Irvine, R. F., 1989. Inositol phosphates and cell signalling. Nature 341, pp.197-205.

Berridge, M. J., Downes, C. P. and Hanley, M. R., 1989. Neural and developmental actions of lithium: a unifying hypothesis. Cell 59, pp. 411-419.

McAllister, G., Whiting, P., Hammond, E. A., Knowles, M., Atack, R., Bailey, J. R., Maigetter, R. and Ragan, C. I., 1992. cDNA cloning of human and rat brain myo-inositol monophosphatase. Expression and characterization of the human recombinant enzyme. Biochem. J. 284, pp. 749-754.

Sjøholt, G., Gulbrandsen, A. K., Løvlie, R., Berle, J. Ø., Molven, A. and Steen, V. M., 2000. A human myo-inositol monophosphatase gene (IMPA2) localized in a putative susceptibility region for bipolar disorder on chromosome 18p11.2: genomic structure and polymorphism screening in manic-depressive patients. Mol. Psychiatry 5, pp. 172-180.

Steen, V. M., Gulbrandsen, A. K., Eiken, H. G. and Berle, J.ø., 1996. Lack of genetic variations in the coding region of the myo-inositol monophosphatase gene in lithium-treated patients with manic depressive illness. Pharmacogenetics 6, pp. 113-116.

Yoon I S, Li P P, Siu K P, Kennedy J L, Cooke R G, Parikh S V, Warsh J J., 2001. Altered IMPA2 gene expression and calcium homeostasis in bipolar disorder. Mol Psychiatry. 6, pp. 678-83.

Yoshikawa, T., Padigaru, M., Karkera, J. D., Sharma, M., Berrettini, W. H., Esterling, L. E. and Detera-Wadleigh, S., 2000. Genomic structure and novel variant of myo-inositol monophosphatase 2 (IMPA2). Mol. Psychiatry 5, pp. 165-171.

1. Use of an isolated IMPA2 protein in an assay to identify anti-anxiety or anti-depression compounds, wherein said compounds are characterized in that they are capable of enhancing neuronal plasticity.

2. Use according to claim 1 wherein the IMPA2 protein is being selected from;
i. mouse IMPA2 (SEQ ID No:4), rat IMPA2 (SEQ ID No:6), human IMPA2 (SEQ ID No:2) or a functional fragment thereof, or
ii. an amino acid sequence encoding an IMPA2 protein, wherein said amino acid sequence has at least 80% sequence identity, preferably at least 90% sequence identity, more preferably at least 95% or most preferably at least 98% sequence identity with the human IMPA2 protein (SEQ ID No:2) over its entire length.

3. Use of an isolated polynucleotide encoding an IMPA2 protein in an assay to identify anti-anxiety or anti-depression compounds, wherein said compounds are characterized in that they are capable of enhancing neuronal plasticity.

4. Use according to claim 3 wherein the isolated polynucleotide encodes an IMPA2 protein in an assay according to the invention, wherein said IMPA2 protein is preferably being selected from;
i. polynucleotides encoding the mouse (EMBL:BC011093—SEQ ID No:3), rat (EMBL:AY160191—SEQ ID No:5) or human (EMBL:BC011093—SEQ ID No:1) IMPA2 enzyme; or
ii. a polynucleotide sequence encodig an IMPA2 protein, wherein said amino acid sequence has at least 80% sequence identity, preferably at least 90% sequence identity, more preferably at least 95% or most preferably at least 98% sequence identity with the polynucleotide encoding for the human IMPA2 protein (SEQ ID No:1) over its entire length.

5. A method to identify anti-anxiety or anti-depression compounds, wherein said anti-anxiety or anti-depression compounds are capable of enhancing neuronal plasticity, said method comprising the steps of:
a) providing a composition comprising an IMPA2 protein;
b) contacting the IMPA2 protein with the test compound; and
c) measuring the activity of the IMPA2 protein wherein a decrease in the IMPA2 activity in the presence of the test compound is an indicator of an anti-anxiety or anti-depression compound.

6. A method for determining whether a compound is capable of enhancing neuronal plasticity, said method comprising the steps of:
a) providing a composition comprising an IMPA2 protein;
b) contacting the IMPA2 protein with the test compound; and
c) measuring the activity of the IMPA2 protein wherein a decrease in the IMPA2 activity in the presence of the test compound is an indicator of a neuronal plasticity enhancing compound.

7. A method according to claim 5 wherein the activity of the IMPA2 protein is assessed by measuring the hydrolysis of myo-inositol 1-phosphate to generate inositol and inorganic phosphate

8. A method according to claim 5 wherein the activity of the IMPA2 protein is assessed by measuring the accumulation of either myo-inositol monophosphate product in the form of radiolabeled inositol or inorganic phosphate (Pi) in the form of radiolabeled ³²Pi or in a colorimetric assay.

9. A method according to claim 5 wherein the compositions comprising the IMPA2 protein could either be cellular extracts, whole cells or organisms expressing the IMPA2 proteins according to the invention.

10. A method of treating a condition associated with an impaired neuronal adaptive response, comprising the step of administering an effective amount of an IMPA2 inhibitor to a subject in need of such treatment.

11. A method according to claim 9 wherein the conditoion associated with an impaired neuronal adaptive response is selected from the group consisting of memory dysfunction or neurodegenerative diseases.

12. A method according to claim 11 wherein the neurodegenerative diseases are selected from the group consisting of senile dementia, Alzheimer's disease, Parkinsosn's disease, Huntington's chorea, cerebellar-spinal adrenoleucodystrophy, pick's disease or Wilson's disease.

13. A method of treating neuronal damage, comprising the step of administering an IMPA2 inhibitor to a subject in need of such treatment.

14. A method according to claim 13, wherein the neuronal damage is selected from the group consisting of stroke, multi-infarct dementia, head trauma, cerebral ischemia, brain injury and neurodegenerative diseases.

15. A method of enhancing memory and learning, comprising the step of administering an effective amount of an IMPA2 inhibitor to a subject in need of such treatment.

16. The use of a compound identified in an assay according to claim 1, in the preparation of a medicament for treating anxiety or in the preparation of a medicament for promoting neuronal plasticity.

17. The use of an IMPA2 knock out animal as a model for neuroplasticity.


SEQUENCE LISTING (18 sequences)

SEQ ID NO: 1 - DNA, 1415 bp, Homo sapiens
  CDS (83)..(949)
ctggcggcgggacggcgggatccggtgggagccggagtcccgccgaggggggctggaggtggaggggcccggcgaggccgcgatgaagccgagcggcgaggaccaggcggcgctggcggccggcccctgggaggagtgcttccaggcggccgtgcagctggcgctgcgggcaggacagatcatcagaaaagcccttactgaggaaaaacgtgtctcaacaaaaacatcagctgcagatcttgtgacagaaacagatcaccttgtggaagatttaattatttctgagttgcgagagaggtttccttcacacaggttcattgcagaagaggccgcggcttctggggccaagtgtgtgctcacccacagcccgacgtggatcatcgaccccatcgacggcacctgcaattttgtgcacagattcccgactgtggcggttagcattggatttgctgttcgacaagagcttgaattcggagtgatttaccactgcacagaggagcggctgtacacgggccggcggggtcggggcgccttctgcaatggccagcggctccgggtctccggggagacagatctctcaaaggccttggttctgacagaaattggccccaaacgtgaccctgcgaccctgaagctgttcctgagtaacatggagcggctgctgcatgccaaggcgcatggggtccgagtgattggaagctccacattggcactctgccacctggcctcaggggccgcggatgcctattaccagtttggcctgcactgctgggatctggcggctgccacagtcatcatcagagaagcaggcggcatcgtgatagacacttcgggtggacccctcgacctcatggcttgcagagtggttgcggccagcacccgggagatggcgatgctcatagctcaggccttacagacgattaactatgggcgggatgatgagaagtgactgcggctgaggcaaagctgctcccaaggcctccctgggctgctgtgggctcctggggaggtggccctcgtggcccacgctccatgccagtggctcacgctctgctcctggctaccccagagggagttgtcacgctacagtgagtggctggccttttaaatcgacgtctctctcaccaggatttggtgtttagctgtttctctctttaatctcacgtagcctttttcaggttagtacgtgttcttctgtcagggccaaaactcaaatctcctgtgaaatacgtattgataatccaatcttgatttttccccccagaatataaatctcaggtaataaggctttagaactgctgataaagcggatcgttctcaggccctccccccggagtacttcagaatgcaataaatcaaaataatggcaaaaaaaaaaaaaaaaaaaaaaaaaaaaaaaaaaaaaaaaaaaaaaa

SEQ ID NO: 2 - protein, 288 aa, Homo sapiens
MKPSGEDQAALAAGPWEECFQAAVQLALRAGQIIRKALTEEKRVSTKTSAADLVTETDHLVEDLIISELRERFPSHRFIAEEAAASGAKCVLTHSPTWIIDPIDGTCNFVHRFPTVAVSIGFAVRQELEFGVIYHCTEERLYTGRRGRGAFCNGQRLRVSGETDLSKALVLTEIGPKRDPATLKLFLSNMERLLHAKAHGVRVIGSSTLALCHLASGAADAYYQFGLHCWDLAAATVIIREAGGIVIDTSGGPLDLMACRVVAASTREMAMLIAQALQTINYGRDDEK

SEQ ID NO: 3 - DNA, 1403 bp, Mus musculus
  CDS (158)..(1030)
cggacgcgtgggcgcgcgctggaggacgcacggcttggcgggaaggcgaggggactgtgcggagcctccggtgctggcggcggccacgagtgggagcccgggtccggccgcagcacgcggaacagagcagaaggtggcggggtccagcgaagccacgatgaagccgagcagcgaggaagagggagagttggtgcagggcgtgggcccctgggatgagtgcttcgaggtggccgtgcagttggcgctgcgtgcgggacaaatcatcaggaaggccctgacagaggaaaaacgcgtctctacaaaaacatctgctgccgatcttgtgacagaaacagatcaccgagtagaagatttaattgtttctgagttgcgaaagcggtttccttcacacaggttcattgcagaagaggccacagcctccggggccaagtgtgtgctcacccacagcccgacctggatcatcgaccccatcgacggcacctgcaacttcgtgcacaggttccccactgtggcagttagcatcggatttgctgttcaccaggagctggaatttggagtgattcaccactgcacagaggagcggctgtacaccggcaggaggggccaaggtgccttttgcaatggccagcggctccaggtctccagggagacagatctcgcaaaggccttggttctgacagaaatcgggcccaaacgcgaccccgataccctgaaagtattcctgagtaacatggagcggctgctgcacgccaaggctcatggcgtccgagtgattggcagctccaccttggccctctgttacctggcctcgggtgcagccgacgcctattatcagtttggccttcactgctgggacctggcagctgccacagtcatcattagagaagcaggcggcattgtgattgacacctcaggtggaccccttgacctcatgtcgtgcagagtcgtagctgctggcaccagagagatggcagtgctcatagctcaggccctacaaactattaactacggccgagacgatgagaagtgagccacacacagctcgaaggctaaaaacgcagcagcaacctgggaaagagctgtcccggtggcttaagttccaggacagtctaccatagctctccctgggccttgcctcggtgcttagctgattctctctaatctcgtgtagccccttttcaggtcggtacgtgttcttttcatcagagccaaacccagatcttgtgaggggtgtgttagtcatccatcctgattgtttttccagaatgcaaatctcaggtgatacagctttagaacgggctctcaggccctcccctgaccgtggtaagacggaatgcaataaatcagaattatagtggtcaaaaaaaaaaaaaaaaaaaaaaaaaaaaaaaaaaaaaaaaaaa

SEQ ID NO: 4 - protein, 290 aa, Mus musculus
MKPSSEEEGELVQGVGPWDECFEVAVQLALRAGQIIRKALTEEKRVSTKTSAADLVTETDHRVEDLIVSELRKRFPSHRFIAEEATASGAKCVLTHSPTWIIDPIDGTCNFVHRFPTVAVSIGFAVHQELEFGVIHHCTEERLYTGRRGQGAFCNGQRLQVSRETDLAKALVLTEIGPKRDPDTLKVFLSNMERLLHAKAHGVRVIGSSTLALCYLASGAADAYYQFGLHCWDLAAATVIIREAGGIVIDTSGGPLDLMSCRVVAAGTREMAVLIAQALQTINYGRDDEK

SEQ ID NO: 5 - DNA, 1341 bp, Rattus norvegicus
  CDS (185)..(1057)
cttgggctgcagccccttgcgggccttcctgctgcaaggcgcgctggaggacgcacggcttggcgggaaggcgaggggaccgtgcggagcctccggtgctggcggcggccaccagttcgggagccgggagccggccgcagcacgcggaacagagcagagggtggcggggcccggcgaaaccacgatgaagccgaacagcgaggaagaggaggagttggtgcagggcgtgggcccctgggacgagtgcttcgaggtggccgtgcagttggcgttgcgtgcgggacaaatcatcagaaaggccctcactgaggaaaaacacgtctcgacgaaaacatctgctgcagatcttgtgacagaaacagatcaccgagtagaagacttaattgtttctgagttgcgaaagcggttcccttcacacaggttcattgcagaagaggccacagcctccggggccaagtgtgtgctcacccacagcccgacctggatcatcgaccccatcgacggcacctgcaactttgtgcacagattccccactgtggcagttagcatcggatttgctgttcaccaggagctggaattcggagtgattcaccactgcacagaggagcggctgtacaccggcaggaggggccagggcgccttttgcaatggccagaggctccaggtctccagggagacagatctcgcaaaggccttggttctgacagaaatcgggcccaaacgtgaccccgatactctgaaagtattcctgagcaacatggagcggctgctgcacgccaaggctcatggggtccgagtgattggcagctccaccttggcgctctgctacttggcctcgggggctgctgatgcctattaccagttcggcctccactgctgggatctggcagctgccacagtcatcatcagagaagcaggtggcattgtgattgacacctcaggtggaccccttgacctcatgtcgtgcagagtggtggctgctggcaccagagagatggcagtgctcatagctcaggccctacaaaccattaactacggccgggacgatgagaagtgagccgtacagagctctaaggctgacatgagcagctccctgggaaagagctgtccaggggcttgagttccgggatagtctaccatagctgtccccggacctcggtgcttagctgatcctctctaatctcgggtagcccctttccaggtcggtacatggtctttcatcagagccaaacccaaatcttgtgaggtgtgttagtcacccatcctggttgttcggaatgcaaatctcaggtaataaagctttagaacgagttctcaggccctcccctgcccgtggtgata

SEQ ID NO: 6 - protein, 290 aa, Rattus norvegicus
MKPNSEEEEELVQGVGPWDECFEVAVQLALRAGQIIRKALTEEKHVSTKTSAADLVTETDHRVEDLIVSELRKRFPSHRFIAEEATASGAKCVLTHSPTWIIDPIDGTCNFVHRFPTVAVSIGFAVHQELEFGVIHHCTEERLYTGRRGQGAFCNGQRLQVSRETDLAKALVLTEIGPKRDPDTLKVFLSNMERLLHAKAHGVRVIGSSTLALCYLASGAADAYYQFGLHCWDLAAATVIIREAGGIVIDTSGGPLDLMSCRVVAAGTREMAVLIAQALQTINYGRDDEK

SEQ ID NO: 7 - DNA, 18 bp, artificial sequence
  IMPA2 forward primer
gaggtggccgtgcagttg

SEQ ID NO: 8 - DNA, 21 bp, Artificial Sequence
  IMPA2 Reverse Primer
agacgcgtttttcctctgtca

SEQ ID NO: 9 - DNA, 23 bp, Artificial Sequence
  IMPA2 Probe
cctgatgatttgtcccgcacgca

SEQ ID NO: 10 - DNA, 22 bp, Artificial Sequence
  IMPA1 Forward primer
agctgtttcaattggcttcctt

SEQ ID NO: 11 - DNA, 23 bp, Artificial sequence
  IMPA1 Reverse primer
gccggtgtacatcttatcttcca

SEQ ID NO: 12 - DNA, 36 bp, Artificial Sequence
  IMP1 Probe
tgaataaagagatggagtttggaattgtgtacagct

SEQ ID NO: 13 - DNA, 18 bp, Artificial Sequence
  MIP Synthase Forward primer
ctgcgccttcctcaatgg

SEQ ID NO: 14 - DNA, 18 bp, Artificial Sequence
  MIP Synthase Reverse Primer
gctgcgaagccagttcca

SEQ ID NO: 15 - DNA, 25 bp, Artificial Sequence
  MIP Synthase Probe
tccccacagaacacactggtacccg

SEQ ID NO: 16 - DNA, 19 bp, Artificial Sequence
  BDNF Forward primer
cgggacggtcacagtccta

SEQ ID NO: 17 - DNA, 26 bp, Artificial Sequence
  BDNF Reverse primer
cacttggtctcgtagaaatactgctt

SEQ ID NO: 18 - DNA, 27 bp, Artificial Sequence
  BDNF Probe
agaaagtcccggtatccaaaggccaac